(19) United States
(12) Patent Application Publication
(10) Pub. No.: US 2022/0079709 A1
(43) Pub. Date: Mar. 17, 2022

(54) Bone Stabilized Base Guide and Method for Attaching and Positioning a Robotic Intra-Oral Surgical Device

(71) Applicant: DDS Company, Inc., Durham, NC (US)

(72) Inventors: Cornelis J. Janse Van Rensburg, Durham, NC (US); Matthew Vrhovac, Durham, NC (US); Richard Meaney, Durham, NC (US); Louis Costanzo, Durham, NC (US)

(73) Assignee: DDS Company, Inc., Durham, NC (US)

(21) Appl. No.: 17/469,435

(22) Filed: Sep. 8, 2021

Related U.S. Application Data
(60) Provisional application No. 63/078,600, filed on Sep. 15, 2020.
(60) Provisional application No. 63/090,907, filed on Oct. 13, 2020.

(51) Int. Cl. A61C 1/08; A61B 34/30

(52) U.S. Cl. CPC A61C 1/084; A61B 34/30

(57) ABSTRACT

A robotic surgical reference guide. The robotic surgical reference guide may include a reference block; a base guide or frame body; and one or more connection arms, wherein the reference block may be connected to the base guide or frame body via the one or more connection arms.

### RELATED APPLICATIONS

This application is related and claims priority to U.S. Provisional Patent Application Nos. 63/078,600, filed on Sep. 15, 2020 and 63/090,907, filed on Oct. 13, 2020, the applications of which are incorporated herein by reference in their entirety.

### TECHNICAL FIELD

The presently disclosed subject matter relates generally to intraoral guided surgery, and more particularly to a bilaterally stabilized, bone-borne base guide and method for attaching and positioning a robotic intra-oral surgical device relative to a patient's anatomy and planned implant placement.

### BACKGROUND

Robotic intraoral implant placement is an emerging technology making use of a hand-piece on a robotic arm held by a surgeon and stabilized by machinery. Generally, a secondary robotic arm is attached to a reference block, establishing position in space. Other surgical systems may index on a reference by use of light or computer vision. The reference block traditionally is attached to a generic tray that sits on the patient's tissue, teeth, or bone and may be fixated by means of pins or screws. This generic tray is not made to fit each individual patient and must be filled with material to stabilize the tray. When fixated, the generic tray or other form of reference will be fastened to the bone at a single site. This unilateral fixation only stabilizes the guide in one area, not across the entire dental arch. Unilateral fixation does little to increase the stability of a reference. This method requires at least two Cone Beam Computed Tomography (CBCT) scans of the patient (with and without the tray in place) to establish the position of the reference block. As the generic tray must sit on some anatomy, this requires the surgeon to work around the tray for full-arch cases.

### SUMMARY

In one embodiment, a robotic surgical reference guide is provided. The robotic surgical reference guide may include a base guide; a reference block; and one or more connection arms, wherein the reference block may be connected to the base guide via the one or more connection arms. The base guide may further include a generally arcuate body portion; one or more connector bodies spaced generally about the body portion; and a plurality of fixation ports spaced generally about the body portion and forming a passage therethrough. The base guide may be configured to index and fixate onto a patient's buccal bone at two or more fixation sites via the plurality of fixation ports. The reference block may be connected to a body portion of the base guide via the one or more connection arms. The reference block may be a platform, stage, or any other suitable type structure. The reference block may be configured to interface with a robotic intra-oral surgical device. The reference block may include a positioning point, wherein the robotic intra-oral surgical device may be configured to interface with the reference block at the positioning point. The base guide may further include protrusions formed on an inner surface of the main body and extending outward therefrom in a generally perpendicular direction. The one or more of the protrusions may be disposed proximate to one or more of the fixation ports.

In another embodiment, a robotic surgical reference guide is provided. The robotic surgical reference guide may include a reference block; a frame body; and one or more connection arms, wherein the reference block may be connected to the frame body via one or more connection arms. The frame body may further include one or more connector bodies formed thereon. The one or more connector bodies may be spaced about a length of the frame body. One or more of the one or more connector bodies may be configured to mate with one or more connector bodies of a base guide. The reference block may be configured to interface with a robotic intra-oral surgical device. The reference block may include a positioning point, wherein the robotic intra-oral surgical device may be configured to interface with the reference block at the positioning point.

In another embodiment, a method of using a robotic surgical reference guide is provided. The method may include providing a robotic surgical reference guide; fixating the robotic surgical reference guide to a patient's anatomy; positioning a robotic surgical device to a reference block of the robotic surgical reference guide; and wherein the robotic surgical reference guide fixates and relates the robotic surgical device to the patient's anatomy in accordance with a defined surgical plan. In one example, the robotic surgical reference guide may include a base guide; a reference block; and one or more connection arms, wherein the reference block may be connected to the base guide via the one or more connection arms. In another example, the robotic surgical reference guide may include a reference block; a frame body; and one or more connection arms, wherein the reference block may be connected to the frame body via one or more connection arms. The method may further include performing a planned surgical procedure. The robotic surgical reference guide may be fixated to the patient's anatomy via a base guide. The base guide may be configured to index and fixate onto the patient's buccal bone at two or more fixation sites via fixation ports formed in the base guide.

### BRIEF DESCRIPTION OF THE DRAWINGS

Having thus described the presently disclosed subject matter in general terms, reference will now be made to the accompanying drawings, which are not necessarily drawn to scale, and wherein:

FIG. 1 illustrates an example of a bone model, planned implants, and a reference block as generated from an example implant planning software;

FIG. 2 illustrates a top perspective view of an example robotic reference attachment (RRA), in accordance with an embodiment of the invention;

FIG. 3 illustrates a side view of an example RRA, in accordance with an embodiment of the invention;

FIG. 4 illustrates a top view of an example RRA indexed on a patient's buccal bone, in accordance with an embodiment of the invention;

FIG. 5 illustrates a side perspective view of an example latched robotic reference attachment (LRRA), in accordance with an embodiment of the invention;

FIG. 6 illustrates a top perspective view of an example LRRA latched into an example base guide, in accordance with an embodiment of the invention;

FIG. 7 illustrates a side perspective view of an example LRRA latched into the base guide, in accordance with an embodiment of the invention;

FIG. 8 illustrates a side view of an example LRRA poised above an example base guide prior to latching, in accordance with an embodiment of the invention;

FIG. 9 illustrates a side view of an example LRRA latched into an example base guide, in accordance with an embodiment of the invention; and

FIG. 10 illustrates a top down view of an example LRRA latched into an example base guide, in accordance with an embodiment of the invention.

### DETAILED DESCRIPTION

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The invention provides a device and method of attaching and, through this attachment, positioning, a robotic intra-oral surgical device (used for oral surgery, placing of implants, drilling osteotomies and/or other intra-oral surgical procedures) in space, in relation to a patient, by affixing at two or more sites to an exposed bony surface of the patient.

The robotic-patient coupling may be achieved by attaching a robotic reference attachment (RRA) or latched robotic reference attachment (LRRA) to a positioning aid (bone borne base guide) connected to a patient's exposed maxillary or mandibular bone. This attachment is generally known as a bone borne or bone stabilized base guide. This fixation may be done a number of different ways, for example, by unilateral key fixation (UKF) (e.g., as disclosed in U.S. patent application Ser. No. 16/869,717 entitled “Tissue Borne Fixation System, Device, and Methods of Making and Using Same”, the disclosure of which is incorporated herein by reference in its entirety), full bone borne guide, partial bone borne guide, tissue depth indicators (TDI's) (e.g., as disclosed in U.S. patent application Ser. No. 16/869,698 entitled “Tissue Borne Stackable Foundation Guide and Method of Making and Using Same”, the disclosure of which is incorporated herein by reference in its entirety), or any other mechanism or technique of bilaterally affixing a base guide to a patient's bony surface.

In one example, a method of attaching and positioning a robotic intra-oral surgical device relative to a patient's anatomy and planned implant placement may include one or more of the following:
1. Patient is scanned with CBCT or other computer topography indexing device.
2. Data is imported in surgical planning software and surgical plan is designed based off computer topography information.
3. The implant placement and/or surgical plan is then related to the patient's bone position, this position will act as the fixed reference position during surgery.
4. A RRA or LRRA block device is designed in the software based off the segmented bone model which is then matched back to the surgical plan. The RRA or LRRA may include a model provided by the surgical equipment manufacturer, a custom model, or any appropriate device to which a robotic intra-oral surgical device may reference for positioning.
5. The RRA or LRRA block device fixates the robotic equipment to the bony anatomy of the patient, and uses this fixed reference point to relate to the surgical site and surgical plan in space.
6. During surgery, the RRA or LRRA block device is attached to the patient's exposed bony anatomy by means of bilateral fixation blocks.
7. The RRA or LRRA block device carries the robotic attachment/link, and once the robotic equipment is attached to the RRA or LRRA block device it relates the robotic surgical device to the patient's anatomy. It should be noted that this robotic link with the RRA or LRRA block device may be physical in nature or may be accomplished by laser, ultrasound, computer vision, or any other system used for positioning in robotic surgery now known or known in the future.

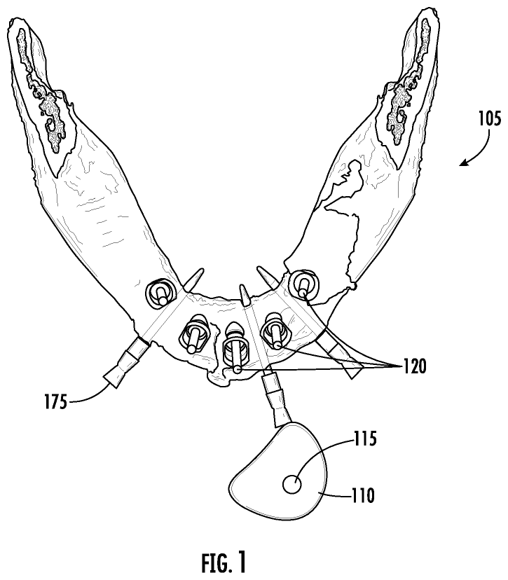

With reference to FIG. 1, in case planning, the surgical plan may include a bone model 105 and a model of a reference block 110 which will be placed during planning. As the relative position of the reference block 110 and one or more planned implant placements 120 may be set digitally, the previously required additional CT scan(s) of the patient as described in the Background section with the generic tray in place is no longer necessary.

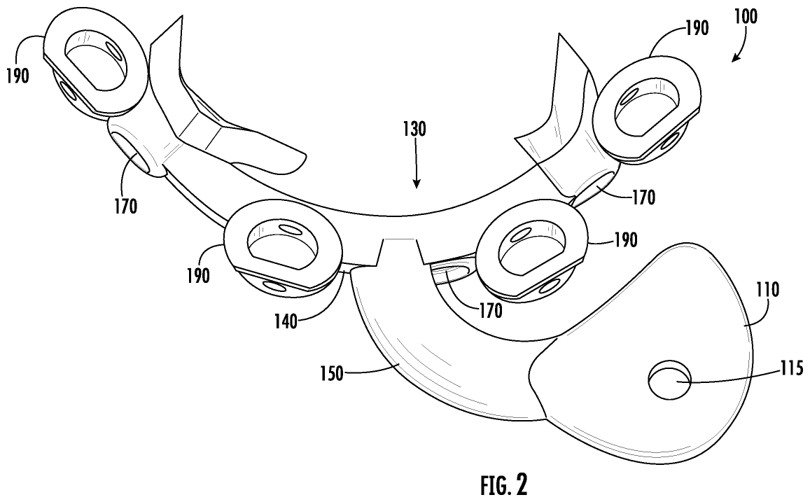

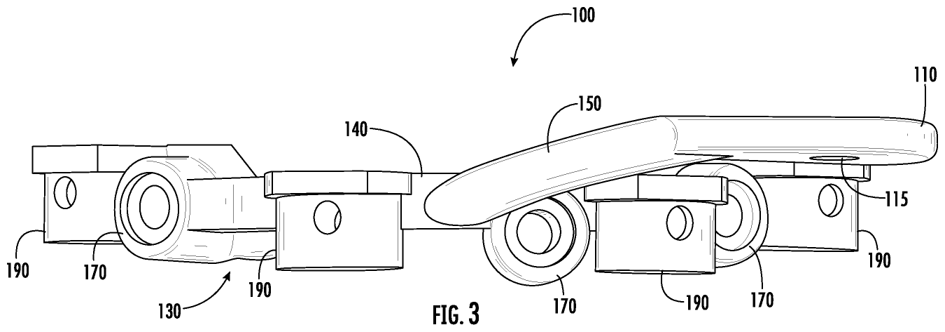

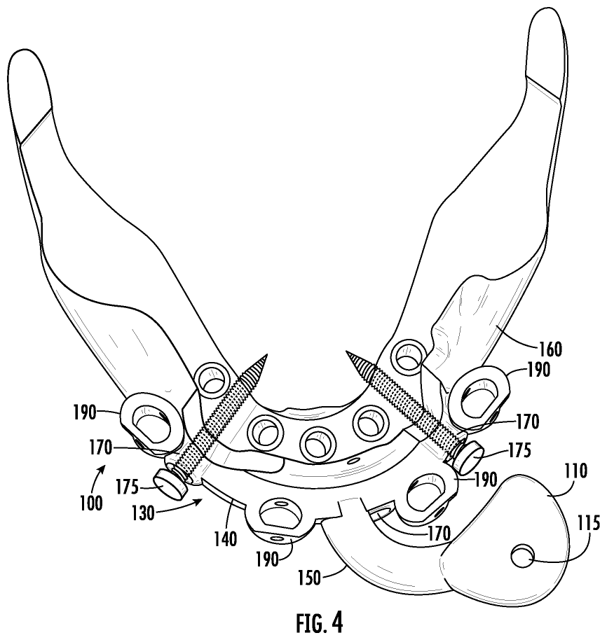

With references to FIGS. 2-4, in one embodiment a base guide 130, such as, for example, a bone borne base guide, that may be common to many full arch guided surgery, may be paired directly with a reference block 110 by a connection arm 150, e.g., a connection arm, protruding from a body portion 140 of the base guide 130. The reference block 110 may be of any design from any manufacturer and is here illustrated by a generic representation. The reference block 110 preferably interfaces/engages with a robotic intra-oral surgical device for positioning data. In one example, the robotic intra-oral surgical device may interface/engage with the reference block 110 at a positioning/attachment point 115.

In one example, the base guide 130 may index on the buccal bone of a patient (e.g., 160), at two or more fixation sites 170, but in other examples may be of the full-buccal, bucco-lingual, or any other type of full-arch base guide. Base guide 130, connection arm 150, and reference block 110 makes up a robotic reference attachment (RRA) guide 100.

The RRA guide 100 may be processed/made with fiber reinforced material, carbon fiber, onyx, Kevlar, printed or milled cobalt chrome, printed or milled titanium, polymers, alloys, printed or milled zirconias, printed resin material, PEEK, CFR PEEK, other printable and/or millable materials, and/or any other technique and/or material suitable for intraoral use now known or known in the future. The RRA guide 100 may be made as a single unitary component.

During surgery, the surgeon may index and fixate the RRA guide 100 directly unto the patient's bone, e.g., buccal bone 160. In one example, the surgeon may fixate the RRA guide 100 directly unto the patient's buccal bone 160 via fixation pins/screws 175 at the two or more fixation sites 170. The surgery may proceed as per the protocol of the guided system being used. In one example, instead of performing osteotomies using a drill guide, the surgeon may attach the robot indexing arm (not shown) to the reference block 110 of the RRA guide 100, and perform osteotomies and implant placement with the help of the robot. In one example, the surgeon may attach the robot indexing arm at positioning/attachment point 115 of the reference block 110 of the RRA guide 100. Once osteotomies and implant placement are complete, the surgery may continue as described in the protocol for the guided system being used.

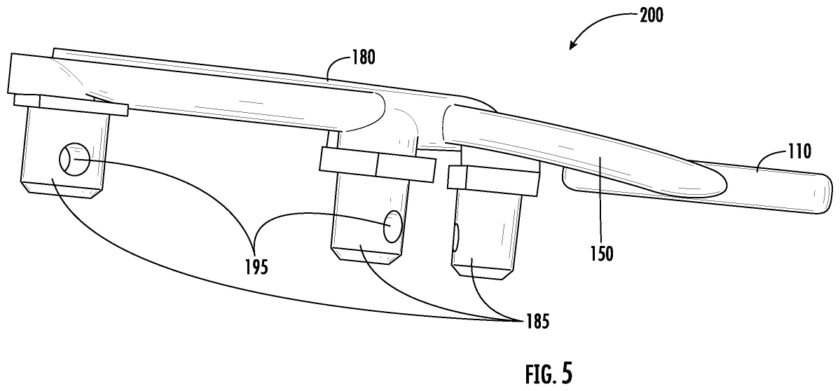

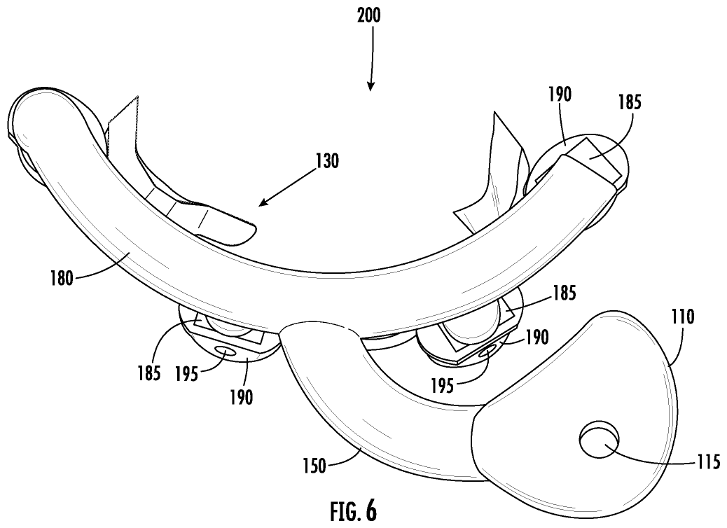

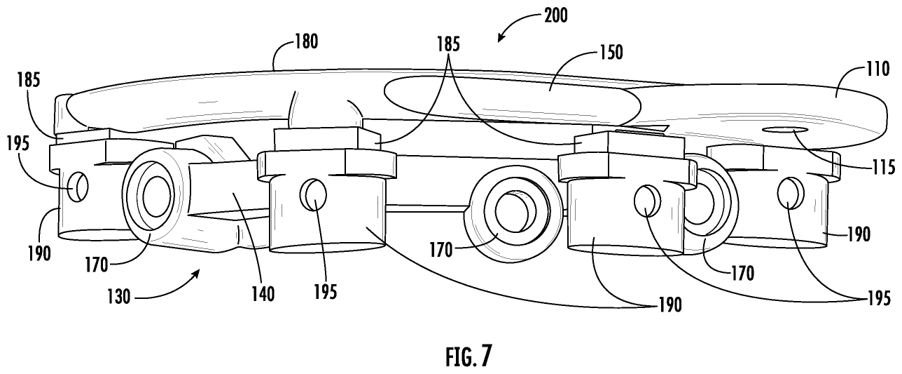

With references to FIGS. 5-7, in another embodiment, reference block 110 may be secured, e.g., latched, in place, so that it can be more easily included in the surgical sequence after seating a base guide, e.g., base guide 130, and then easily removed so the surgery can be finished per the protocol in use. A framework 180 may include connectors 185, such as ‘plug’ latches. The reference block 110 may be connected to the framework 180 via connection arm 150. The reference block 110, connection arm 150, framework 180, and connectors 185 make up the latched robotic reference attachment (LRRA) 200. In this example, the LRRA 200 may be securely connected to connectors 190, such as ‘jack’ latches, of a base guide 130, e.g., the LRRA 200 may be latched into ‘jack’ latches 190, of base guide 130. In one example, the ‘plug’ latches 185 and ‘jack’ latches 190 both include corresponding coupling pin holes 195, and the LRRA 200 may be latched into the ‘jack’ latches 190 of the base guide 130 by inserting coupling pins (not shown) into the coupling pin holes, thereby securing the LRRA 200 in place with the base guide 130. The LRRA 200 may replace any type of osteotomy guide as the robotic system may be used to drill osteotomies and place implants. However, a backup osteotomy guide may be provided in case of equipment failure.

During surgery, when preparing for osteotomies and implant placement, the surgeon may latch and secure the LRRA 200 to the base guide, such as base guide 130, perform the drilling and implant placement with the robotic arm, and then remove the LRRA 200 from the base guide 130. The surgeon may then proceed as per the guided surgery protocol being used.

The LRRA 200 may be processed/made with fiber reinforced material, carbon fiber, onyx, Kevlar, printed or milled cobalt chrome, printed or milled titanium, polymers, alloys, printed or milled zirconias, printed resin material, PEEK, CFR PEEK, other printable and/or millable materials, and/or any other material suitable for intraoral use now known or known in the future. The LRRA guide 200 may be made as a single unitary component.

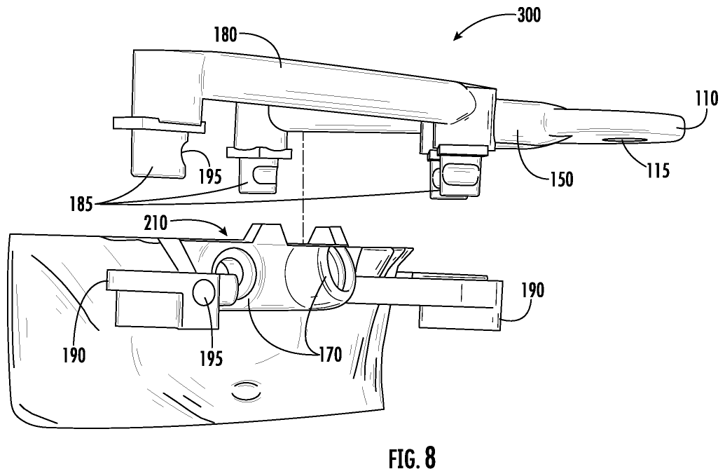

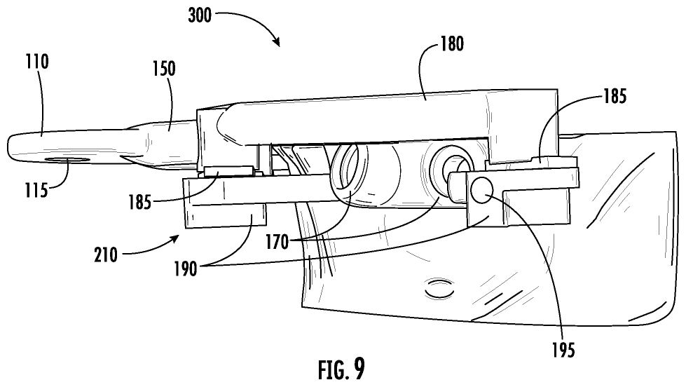

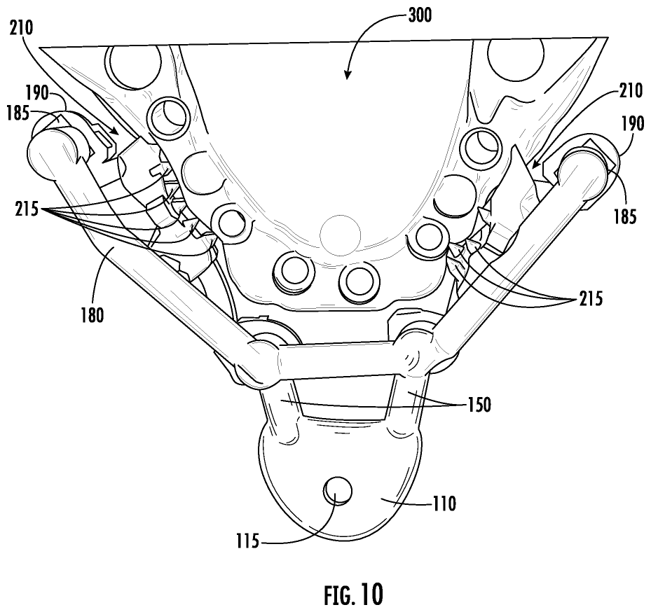

With reference to FIGS. 8-10, in another embodiment and making use of a device, such as, for example, that described in U.S. patent application Ser. No. 16/869,698 entitled “Tissue Borne Stackable Foundation Guide and Method of Making and Using Same”, for example, reference block 110 may be secured, e.g., latched in place, so it can be more readily included in the surgical sequence after seating the guide 210, e.g., a tissue borne stackable guide, and then easily removed so the surgery can be finished per the protocol in use. A framework 180 may include connectors 185, such as ‘plug’ latches. The reference block 110 may attach to the framework 180. In one example, reference block 110 maybe connected to the framework 180 via connection arms 150. The reference block 110, framework 180, connection arms 150, and connectors 185 make up the latched robotic reference attachment (LRRA) 300. In this example, the LRRA 300 may be securely attached, e.g., latched, into connectors 190, such as ‘jack’ latches, on the guide 210. In one example, the connectors 1 ‘plug’ latches 185 and ‘jack’ latches 190 both include corresponding coupling pin holes 195, and the LRRA 200 may be latched into the ‘jack’ latches 190 of the base guide 130 by inserting coupling pins (not shown) into the coupling pin holes, thereby securing the LRRA 300 in place with the base guide 130. The LRRA 300 may replace any type of osteotomy guide as the robotic system will be used to drill osteotomies and place implants. However, a backup osteotomy guide may be provided in case of equipment failure.

During surgery, when preparing for osteotomies and implant placement, the surgeon may latch and secure the LRRA 300 to the guide 210, perform the drilling and implant placement with the robotic arm, and then remove the LRRA 300 from the guide 210. The surgeon may then proceed as per the guided surgery protocol being used.

The LRRA 300 may be processed/made with fiber reinforced material, carbon fiber, onyx, Kevlar, printed or milled cobalt chrome, printed or milled titanium, polymers, alloys, printed or milled zirconias, printed resin material, PEEK, CFR PEEK, other printable and/or millable materials, and/or any other material suitable for intraoral use now known or known in the future. The LRRA guide 300 may be made as a single unitary component.

In one example, guide 130 and/or guide 210 may include tissue depth indicators (TDI's) 215. Tissue thickness indictors 215 may be protrusions, and in one example, these protrusions protrude in a generally perpendicular direction out from an inner surface of the base guide 130 and/or guide 210.

The RRA 100, LRRA 200, and LRRA 300 devices and methods improve on existing intraoral robotic surgery systems by requiring only one CBCT scan, as current systems require multiple CT scans. This method greatly reduces the need to expose the patient to unnecessary radiation.

The RRA 100, LRRA 200, and LRRA 300 devices and methods also improve surgical stability by bilaterally anchoring securely to the patient's bony anatomy. Current devices use a generic (base guide) device which attaches via pin or screw, but may not engage the bony anatomical surface. Any current device that fixates to the patient's bony anatomy does so at a single site and so does not greatly improve surgical stability. Any current device that fixates over the patient's tissue risks necrosis due to pressure on the mucosa. By making use of the tissue-borne, bone-stabilized device, such as that described in “Tissue Borne Stackable Foundation Guide and Method of Making and Using Same,” this risk can be minimized. The RRA 100 and LRRA 200 devices are patient specific custom designed, bilateral, bone fixation reference base guides.

The RRA 100, LRRA 200, and LRRA 300 devices leave the surgical site accessible by attaching securely onto the patient's bony anatomy away from the actual surgical site.

The RRA 100, LRRA 200, and LRRA 300 devices and methods may utilize the devices and methods disclosed in either of U.S. patent application Ser. No. 16/869,717 entitled “Tissue Borne Fixation System, Device, and Methods of Making and Using Same” or U.S. patent application Ser. No. 16/869,698 entitled “Tissue Borne Stackable Foundation Guide and Method of Making and Using Same”, the disclosures of which are both incorporated herein by reference in their entirety), as non-limiting examples of compatible systems, but it should be noted that the RRA 100, LRRA 200, and LRRA 300 devices and methods are applicable to any suitable full-arch guided surgery system.

The RRA 100, LRRA 200, and LRRA 300 devices and methods utilize a bilaterally stabilized bone-borne device for robotic implant guidance, decreasing the number of unnecessary CBCT scans, solidly affixing the reference block to the patient's existing bony anatomy, and stabilizing the guide across the dental arch.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The subject treated by the presently disclosed devices and methods in their many embodiments is desirably a human subject, although it is to be understood that the methods described herein are effective with respect to all vertebrate species, which are intended to be included in the term “subject.”

A “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A robotic surgical reference guide, comprising:
a. a base guide;
b. a reference block; and
c. one or more connection arms, wherein the reference block is connected to the base guide via the one or more connection arms.

2. The reference guide of claim 1, wherein the base guide comprises:
a. a generally arcuate body portion;
b. one or more connector bodies spaced generally about the body portion; and
c. a plurality of fixation ports spaced about the body portion and forming a passage therethrough.

3. The reference guide of claim 2, wherein the base guide is configured to index and fixate onto a patient's buccal bone at two or more fixation sites via the plurality of fixation ports.

4. The reference guide of claim 1, wherein the reference block is connected to a body portion of the base guide via the one or more connection arms.

5. The reference guide of claim 1, wherein the reference block is configured to interface with a robotic intra-oral surgical device.

6. The reference guide of claim 5, wherein the reference block comprises a positioning point, wherein the robotic intra-oral surgical device is configured to interface with the reference block at the positioning point.

7. The reference guide of claim 1, wherein the base guide further comprises protrusions formed on an inner surface of the main body and extending outward therefrom in a generally perpendicular direction.

8. The reference guide of claim 7, wherein the one or more of the protrusions are disposed proximate to one or more of the fixation ports.

9. A robotic surgical reference guide, comprising:
a. a reference block;
b. a frame body; and
c. one or more connection arms, wherein the reference block is connected to the frame body via one or more connection arms.

10. The reference guide of claim 9, wherein the frame body further comprises one or more connector bodies formed thereon.

11. The reference guide of claim 10, wherein the one or more connector bodies are spaced about a length of the frame body.

12. The reference guide of claim 10, wherein one or more of the one or more connector bodies are configured to mate with one or more connector bodies of a base guide.

13. The reference guide of claim 9, wherein the reference block is configured to interface with a robotic intra-oral surgical device.

14. The reference guide of claim 13, wherein the reference block comprises a positioning point, wherein the robotic intra-oral surgical device is configured to interface with the reference block at the positioning point.

15. A method of using a robotic surgical reference guide, the method comprising:
a. providing a robotic surgical reference guide;
b. fixating the robotic surgical reference guide to a patient's anatomy;
c. positioning a robotic surgical device to a reference block of the robotic surgical reference guide; and
wherein the robotic surgical reference guide fixates and relates the robotic surgical device to the patient's anatomy in accordance with a defined surgical plan.

16. The method of claim 15, wherein the robotic surgical reference guide comprises:
a. a base guide;
b. a reference block; and
c. one or more connection arms, wherein the reference block is connected to the base guide via the one or more connection arms.

17. The method of claim 15, wherein the robotic surgical reference guide comprises:
a. a reference block;
b. a frame body; and
c. one or more connection arms, wherein the reference block is connected to the frame body via one or more connection arms.

18. The method of claim 15, further comprising performing a planned surgical procedure.

19. The method of claim 15, wherein the robotic surgical reference guide is fixated to the patient's anatomy via a base guide.

20. The method of claim 19, wherein the base guide is configured to index and fixate onto the patient's buccal bone at two or more fixation sites via fixation ports formed in the base guide.
